(19) United States
(12) Patent Application Publication
(10) Pub. No.: US 2013/0108567 A1
(43) Pub. Date: May 2, 2013

(54) SKIN-WHITENING COMPOSITION FOR EXTERNAL APPLICATION ON SKIN CONTAINING EXTRACTS FROM PAPER MULBERRY FLOWERS AND FRUITS

(71) Applicants: Jin Young Lee, Yongin-si (KR); Hyung Jun Lim, Yongin-si (KR)

(72) Inventors: Jin Young Lee, Yongin-si (KR); Hyung Jun Lim, Yongin-si (KR)

(73) Assignee: AMOREPACIFIC CORPORATION, Seoul (KR)

(21) Appl. No.: 13/807,904

(22) PCT Filed: Jul. 4, 2011
(86) PCT No.: PCT/KR2011/004885

§ 371 Date: Dec. 31, 2012

(30) Foreign Application Priority Data
Jul. 2, 2010 (KR) 10-2010-0063992

(51) Int. Cl. A61K 8/97; A61Q 19/02

(52) U.S. Cl. CPC A61K 8/97; A61Q 19/02
USPC 424/62

(57) ABSTRACT

The present invention relates to a skin external composition comprising an extract of the flower and fruit of paper mulberry, and more particularly to a skin external composition containing, as an active ingredient, an extract of flower and fruit of paper mulberry, which shows an excellent skin-whitening effect by effectively inhibiting melanin production.

### TECHNICAL FIELD

The present invention relates to a skin external composition comprising an extract of the flower and fruit of paper mulberry, and more particularly to a skin external composition comprising, as an active ingredient, an extract of flower and fruit of paper mulberry, which shows an excellent skin-whitening effect by effectively inhibiting melanin production.

### BACKGROUND ART

Human skin color is determined by various factors, including the activity of melanocytes which produce the melanin pigment, distribution of blood vessels, skin thickness, the presence or absence of pigments such as carotenoids and bilirubin, or the like. Of the factors, the dark pigment melanin which is produced from melanocytes by the action of various enzymes including tyrosinase is the most important. The production of melanin is affected by genetic factors, physiological factors such as hormone secretion, stress, etc. and environmental factors such as UV radiation. Melanin present in the skin plays an important role in protecting the skin from UV, etc. However, it is known that excessive production of melanin plays major roles in accelerating pigmentation and skin aging and inducing skin cancer. From long ago, ascorbic acid, kojic acid, arbutin, hydroquinone, glutathione, their derivatives, or compounds having tyrosinase inhibitory activity have been used in cosmetics or drugs in order to treat or ameliorate anomalous skin pigmentation. and excessive melanin pigmentation caused by exposure to UV. However, their use is restricted. because of insufficient skin whitening effect, safety issue on the skin, formulation instability when used in cosmetics, or the like.

### DISCLOSURE

### Technical Problem

Accordingly, the present inventors have conducted studies on natural materials having high skin-whitening effects and, as a result, have found that an extract of the flower and fruit of paper mulberry has an excellent skin-whitening effect, thereby completing the present invention.

Therefore, it is an object of the present invention to provide a skin external composition comprising a natural material which has an excellent skin-whitening effect and is safe for the human body.

### Technical Solution

In order to accomplish the above object, the present invention provides a skin external composition for skin whitening comprising an extract of the flower and fruit of paper mulberry as an active ingredient.

### Advantageous Effects

The skin external composition according to the present invention comprises, as an active ingredient, an extract of the flower and fruit of paper mulberry. The extract according to the present invention exhibits an excellent skin-whitening effect by effectively inhibiting melanin production, compared to extracts of other portions (including leaf, stem and root) of paper mulberry and is safe for use on the skin.

### Best Mode

The present invention provides a skin external composition for skin whitening comprising an extract of the flower and fruit of paper mulberry as an active ingredient.

Hereinafter, the present invention will be described in detail.

Plants belonging to the genus Broussonetia (paper mulberry) which is used as an active ingredient in the present invention include Broussonetia kazinoki Sieb, Broussonetia papyrifera Vent and the like. These plants are deciduous broad-leaf shrubs that are distributed in most areas of Korea (mainly the southern part), China, Taiwan, Japan and the like and. grow in. sunny places of mountains, places around fields, etc. The bast fiber of paper mulberry has been used as a raw material for making paper, and it is known that paper mulberry has various medicinal effects, including tonic, eyesight improvement, impotence alleviation, dropsy treatment, revivification, diuresis, palsy removal, augmentation, blood clarification, amblyopia treatment, and the like.

The extract of the flower and fruit of paper mulberry according to the present invention can be obtained, according to any method known in the art. For example, the extract can be prepared by drying the flower and fruit of paper mulberry in sunlight or hot air and extracting the dried flower and fruit.

The skin external composition according to the present invention comprises an extract of the flower and fruit of paper mulberry. The extract according to the present invention exhibits an excellent skin-whitening effect by effectively inhibiting melanin production, compared to extracts of other portions (including leaf, stem and root) of paper mulberry, when it is applied to the skin. Thus, the extract according to the present invention can be applied to a skin external composition for skin whitening.

The skin external composition for skin whitening according to the present invention may comprise the extract of the flower and fruit of paper mulberry in an amount of 0.0001-50 wt% based on the total weight of the composition depending on the formulation of the composition. If the content of the extract in the composition is less than 0.0001 wt%, the skin-whitening effect of the composition will be insignificant, and if the content is more than 50 wt%, the stability of the formulation will be poor.

The composition according to the present invention may contain a cosmetically and dermatologically acceptable medium or base. The composition may be formulated as a preparation for topical application. Examples of formulations for local application include solution, gel, solid or dough anhydride, emulsion prepared by dispersing oil phase in a water phase, suspension, microemulsion, microcapsule, microgranule, ionic (liposome) and/or non-ionic vesicle, cream, skin, lotion powder, spray, and conceal stick. Also, the skin external composition according to the present invention can be formulated as a foam composition or an aerosol composition further containing a compressed propellant. In addition, the composition according to the present invention can be prepared formulated according to a conventional method known in the art.

The skin whitening composition according to the present invention may contain additives which are conventionally field in the cosmetic field or the skin science field, for example, fatty substance, organic solvent, resolvent, thickener, gelling agent, softener, antioxidant, suspending agent, stabilizer, foaming agent, fragrance ingredient, surfactant, water, ionic or non-ionic emulsifying agent, filler, sequestering agent, chelating agent, preservative, vitamins, blocker, wetting agent, essential oil, dye, pigment, hydrophilic or hydrophobic activator, lipid vesicle or other components. These additives are contained in amounts which are generally used in the cosmetic field or the skin science field.

Hereinafter, the present invention will be described in further detail with reference to examples and test examples. It is to be understood, however, that these examples are for illustrative purposes only and are not intended to limit the scope of the present invention.

### MODE FOR INVENTION

### Preparation Example 1

### Preparation of Extract of Flower and Fruit of Paper Mulberry

1 kg of the flower and fruit of paper mulberry were added to 10 L of clean water and extracted by boiling in an extractor equipped with a cooling condenser for 5 hours. The extract was filtered through a 300-mesh filter cloth and allowed to stand at 5 to 15° C. for 5 days, after which it was filtered through filter paper. The filtrate was concentrated under reduced pressure in a distillation device system with a cooling condenser, thereby obtaining 70 g of an extract of the flower and fruit of paper mulberry.

### Preparation Example 2

### Preparation of Extract of Leaf, Stem and Root of Paper Mulberry

1 kg of the leaf, stem and root of paper mulberry were added to 10 L of clean water and extracted by boiling in an extractor equipped with a cooling condenser for 5 hours. The extract was filtered through a 300-mesh filter cloth and allowed to stand at 5 to 15° C. for 5 days, after which it was filtered through filter paper. The filtrate was concentrated under reduced pressure in a distillation device system with a cooling condenser, thereby obtaining 70 g of an extract of the leaf, stem and root of paper mulberry.

### Test Example 1

### Tyrosinase Inhibitory effect

The enzyme tyrosinase used in this Test Example was a tyrosinase (SIGMA) extracted from mushrooms. First, the substrate tyrosine was dissolved in distilled water at a concentration of 0.3 mg/ml, and 1.0 ml of the solution was placed in each test tube. Then, 1.0 ml of potassium-phosphate buffer (0.1 M, pH 6.8) and 0.7 ml of distilled water were added thereto, thereby preparing a mixed solution.

Each of the extracts of Preparation Examples 1 and 2 was dissolved in ethanol at a suitable concentration, and of each of the extract sample solutions was added to the mixed solution, and then reacted to react in an incubator at 37° C. for 10 minutes. As a control, 0.2 ml of a solvent alone was used instead of each extract sample. 0.1 ml of a tyrosinase solution (2500 unit/ml) was added to each of the reaction solutions and allowed to react in an incubator at 37° C. for 10 minutes. Then, the test tube containing the reaction solution was quenched in ice water to stop the reaction, and the absorbance at 475 nm was measured with a photoelectric spectrophotometer. Then, the tyrosinase inhibitory activity of each of the extracts was calculated using the following equation 1, and the results of the calculation are shown in Table 1 below as the extract concentration (IC₅₀) that inhibited the enzyme activity by 50%.

Tyrosinase activity inhibition (%)=[(A-B)/A]×100 [Equation 1]

wherein A: absorbance of test tube containing no test material; and

B: absorbance of test tube containing test material.

TABLE 1

| | Tyrosinase inhibitory |
|---|---|
| Test material | activity (IC₅₀) |
| Extract of flower and fruit of paper | 1 μg/ml |
| mulberry (Preparation Example 1) | |
| Extract of leaf, stem and root of paper | 2.5 μg/ml |
| mulberry (Preparation Example 2) | |

As can be seen from the results in Table 1 above, the extract of the flower and fruit of paper mulberry according to the present invention more effectively inhibited melanin production compared to the extract of the leaf, stem and root of paper mulberry.

### Example and Comparative Examples 1 and 2

Lotion formulations of Example and Comparative Examples 1 and 2 were prepared using the compositions shown in Table 2 below (unit: wt%).

TABLE 2

| | | Comparative | Comparative |
|---|---|---|---|
| Component | Example | Example 1 | Example 2 |
| 1. Cetearyl alcohol | 1.0 | 1.0 | 1.0 |
| 2. Lipophilic glyceryl stearate | 1.0 | 1.0 | 1.0 |
| 3. Glyceryl stearate SE | 1.5 | 1.5 | 1.5 |
| 4. Phytosqualane | 3 | 3 | 3 |
| 5. Hydeogenated polydecene | 2 | 2 | 2 |
| 6. Dimethicone | 0.5 | 0.5 | 0.5 |
| 7. Polysorbate 60 | 1 | 1 | 1 |
| 8. Sorbitan sesquioleate | 0.4 | 0.4 | 0.4 |
| 9. Methylparaben | 0.1 | 0.1 | 0.1 |
| 10. Propylparaben | 0.05 | 0.05 | 0.05 |
| 11. Purified water | To 100 | To 100 | To 100 |
| 12. Butylene glycol | 5 | 5 | 5 |
| 13. Polyacrylate-13* | 0.5 | 0.5 | 0.5 |
| polyisobutene * polysorbate 20 | | | |
| 14. Extract of flower and fruit | 1 | — | — |
| of paper mulberry | | | |
| (Preparation Example 1) | | | |
| 15. Extract of leaf, stem and | — | 1 | — |
| root of paper mulberry | | | |
| (Preparation Example 2) | | | |

### Method for Preparation of Formulations of Examples and Comparative Examples 1 and 2

1) Components 11 to 15 were uniformly mixed with each other while they were heated to 70° C., thereby preparing an aqueous phase.

2) Components 1 to 10 were uniformly mixed with each other while they were heated to 70° C., thereby preparing an oil phase.

3) The oil phase of 2) was added to the aqueous phase 1) and homomixed at 7,200 rpm for 6 minutes.

4) The mixture of 3) was cooled to room temperature.

### Test Example 2

### Test for Skin Whitening Effect on Human Skin

An opaque tape having a perforation (1.5 cm (W)×1.5 cm (L)) was attached to the upper arm portion of each of 12 healthy men, and then the attached portion was radiated with UVB at a dose about 1.5-2 times the minimal erythema dose of each subject to induce skin darkening.

After the UV radiation, each of the formulations of Examples and Comparative Examples 1 and 2 was applied on the UV-radiated portion, and a control portion (not applied with anything) was provided. The change in the state of the skin was observed over 8 weeks. At 4, 6 and 8 weeks, the color of the skin was measured using the colorimeter CR2002 (Japan, Minolta). The difference (ΔL*) in skin color between the time point of initiation of application and the time point of completion of application of each formulation was calculated according to the following equation 2, and the calculation results are shown in Table 3 below. Meanwhile, the whitening effect is evaluated by comparing the ΔL* value between the sample-applied portion and the control portion.

ΔL*=L* value at time point of measurement−L* value after induction of darkening at initial stage of measurement [Equation 2]

TABLE 3

| | Lightness (ΔL*) of skin color | | |
|---|---|---|---|
| | 4 weeks | 6 weeks | 8 weeks |
| Example | 1.4 | 1.7 | 1.8 |
| Comparative | 1.1 | 1.6 | 1.7 |
| Example 1 | | | |
| Comparative | 0.2 | 0.6 | 1.2 |
| Example 2 | | | |

As can be seen in Table 3 above, the human skin-whitening effect of the Example containing the extract of the flower and fruit of paper mulberry was much higher than that of Comparative Example 2 containing no paper mulberry extract and was also significantly higher than that of Comparative Example 1 containing the extract of the leaf, stem and root of paper mulberry.

### Formulation Example 1

### Milk Lotion

Milk lotion was prepared using the composition shown in Table 4 below according to conventional method.

TABLE 4

| Component | Content (wt %) |
|---|---|
| Purified water | Balance |
| Glycerin | 8.0 |
| Butylene glycol | 4.0 |
| Hyaluronic acid extract | 5.0 |
| Beta-glucan | 7.0 |
| Carbomer | 0.1 |
| Extract of flower and fruit of | 0.05 |
| paper mulberry | |
| Caprylic/capric triglyceride | 8.0 |
| Squalane | 5.0 |
| Cetearyl glucoside | 1.5 |
| Sorbitan stearate | 0.4 |
| Cetearyl alcohol | 1.0 |
| Preservative | q.s. |
| Fragrance | q.s. |
| Pigment | q.s. |
| Triethanolamine | 0.1 |

### Formulation Example 2

### Nourishing lotion

Nourishing lotion was prepared using the composition shown in Table 5 below according to conventional method.

TABLE 5

| Component | Content (wt %) |
|---|---|
| Purified water | Balance |
| Glycerin | 3.0 |
| Butylene glycol | 3.0 |
| Liquid paraffin | 5.0 |
| Beta-glucan | 7.0 |
| Carbomer | 0.1 |
| Extract of flower and fruit of | 3.0 |
| paper mulberry | |
| Caprylic/capric triglyceride | 3.0 |
| Squalane | 5.0 |
| Cetearyl glucoside | 1.5 |
| Sorbitan stearate | 0.4 |
| Polysorbate 60 | 1.5 |
| Preservative | q.s. |
| Fragrance | q.s. |
| Pigment | q.s. |
| Triethanolamine | 0.1 |

### Formulation Example 3

### Nourishing Cream

Nourishing cream was prepared using the composition shown in Table 6 below according to conventional method.

TABLE 6

| Component | Content (wt %) |
|---|---|
| Purified water | Balance |
| Glycerin | 3.0 |
| Butylene glycol | 3.0 |
| Liquid paraffin | 7.0 |
| Bata-glucan | 7.0 |
| Carbomer | 0.1 |
| Extract of flower and fruit of | 3.0 |
| paper mulberry | |
| Caprylic/capric triglyceride | 3.0 |
| Squalane | 5.0 |
| Cetearyl glucoside | 1.5 |
| Sorbitan stearate | 0.4 |
| Polysorbate 60 | 1.2 |
| Preservative | q.s. |
| Fragrance | q.s. |
| Pigment | q.s. |
| Triethanolamine | 0.1 |

### Formulation Example 4

### Pack

A pack was prepared using the composition shown in Table 7 below according to conventional method.

TABLE 7

| Component | Content (wt %) |
|---|---|
| Purified water | Balance |
| Glycerin | 4.0 |
| Polyvinyl alcohol | 15.0 |
| Hyaluronic acid extract | 5.0 |
| Beta-glucan | 7.0 |
| Allantoin | 0.1 |
| Extract of flower and fruit of | 0.5 |
| paper mulberry | |
| Nonylphenyl ether | 0.4 |
| Polysorbate 60 | 1.2 |
| Preservative | q.s. |
| Fragrance | q.s. |
| Pigment | q.s. |
| Ethanol | 6.0 |

### Formulation Example 5

### Ointment

An ointment was prepared using the composition shown in Table 8 below according to conventional method.

TABLE 8

| Component | Content (wt %) |
|---|---|
| Purified water | Balance |
| Glycerin | 8.0 |
| Butylene glycol | 4.0 |
| Liquid paraffin | 15.0 |
| Beta-glucan | 7.0 |
| Carbomer | 0.1 |
| Extract of flower and fruit of | 1.0 |
| paper mulberry | |
| Caprylic/capric triglyceride | 3.0 |
| Squalane | 1.0 |
| Cetearyl glucoside | 1.5 |
| Sorbitan stearate | 0.4 |
| Polysorbate 60 | 1.0 |
| Preservative | q.s. |
| Fragrance | q.s. |
| Pigment | q.s. |
| Beeswax | 4.0 |

1. A skin external composition for skin whitening comprising an extract of flower and fruit of paper mulberry as an active ingredient.

2. The skin external composition of claim 1, wherein the composition comprises the extract of flower and fruit of paper mulberry in an amount of 0.0001-50.0 wt% based on the total weight of the composition.
